(19) United States
(12) Patent Application Publication
(10) Pub. No.: US 2024/0156516 A1
(43) Pub. Date: May 16, 2024

(54) SURGICAL INSTRUMENTS, SYSTEMS, AND METHODS INCORPORATING ELECTROSURGICAL FUNCTIONALITY FOR AN ULTRASONIC BLADE

(71) Applicant: Covidien LP, Mansfield, MA (US)

(72) Inventors: Thomas E. Drochner, Longmont, CO (US); Matthew S. Cowley, Frederick, CO (US); Kenlyn Bonn, Lakewood, CO (US); James R. Fagan, Erie, CO (US); Michael B. Lyons, Boulder, CO (US); David J. Van Tol, Boulder, CO (US)

(21) Appl. No.: 18/282,267

(22) PCT Filed: Mar. 7, 2022
(86) PCT No.: PCT/IB2022/052004

§ 371 Date: Sep. 15, 2023

Related U.S. Application Data
(60) Provisional application No. 63/162,247, filed on Mar. 17, 2021.

(51) Int. Cl. A61B 18/14

(52) U.S. Cl. CPC A61B 18/1447; A61B 2017/320094

(57) ABSTRACT

An end effector assembly of a surgical instrument includes an ultrasonic blade adapted to receive ultrasonic energy from a source of ultrasonic energy to vibrate the ultrasonic blade. A jaw member is movable relative to the ultrasonic blade from a spaced-apart position to an approximated position for clamping tissue. A jaw liner is engaged with the jaw member such that the jaw liner contacts the ultrasonic blade when the jaw member is in the approximated position. The ultrasonic blade is adapted to receive electrosurgical energy from a source of electrosurgical energy. The ultrasonic blade defines a distal tip. The distal tip of the ultrasonic blade is configured to direct electrosurgical energy.

### CROSS-REFERENCE TO RELATED APPLICATIONS

This application is a 371 National Stage Application of International Application No. PCT/IB2022/052004, filed Mar. 7, 2022, which claims the benefit of, and priority to, U.S. Provisional Patent Application No. 63/162,247, filed on Mar. 17, 2021, the entire contents of which are hereby incorporated herein by reference.

### FIELD

The present disclosure relates to energy-based surgical instruments and, more particularly, to surgical instruments, systems, and methods incorporating electrosurgical functionality to an ultrasonic blade to facilitate energy-based tissue treatment.

### BACKGROUND

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to treat tissue. An ultrasonic surgical device may include, for example, an ultrasonic blade and a clamp mechanism to enable clamping of tissue against the blade. Ultrasonic energy transmitted to the blade causes the blade to vibrate at very high frequencies, which allows for heating tissue to treat tissue clamped against or otherwise in contact with the blade.

Electrosurgical instruments and systems conduct Radio Frequency (RF) energy through tissue to treat tissue. An electrosurgical instrument or system may be configured to conduct bipolar RF energy between oppositely charged electrodes and through tissue, e.g., tissue clamped between the electrodes or otherwise in contact therewith, to treat tissue. Alternatively, or additionally, an electrosurgical instrument or system may be configured to deliver monopolar RF energy from an active electrode to tissue in contact with the electrode, with the energy returning via a remote return electrode device to complete the circuit.

### SUMMARY

Provided in accordance with aspects of the disclosure is an end effector assembly of a surgical instrument including an ultrasonic blade adapted to receive ultrasonic energy from a source of ultrasonic energy to vibrate the ultrasonic blade. A jaw member is movable relative to the ultrasonic blade from a spaced-apart position to an approximated position for clamping tissue. A jaw liner is engaged with the jaw member such that the jaw liner contacts the ultrasonic blade when the jaw member is in the approximated position. The ultrasonic blade is adapted to receive electrosurgical energy from a source of electrosurgical energy. The ultrasonic blade defines a distal tip.

According to aspects of the disclosure, the distal tip of the ultrasonic blade includes a tapered edge defining a pointed distal end.

According to aspects of the disclosure, the ultrasonic blade is adapted for monopolar treatment.

According to aspects of the disclosure, the ultrasonic blade is adapted to receive ultrasonic energy and electrosurgical energy simultaneously.

According to aspects of the disclosure, a portion of the ultrasonic blade includes an insulative coating adapted to direct monopolar energy. The insulative coating may be a nonstick coating.

According to aspects of the disclosure, the distal tip of the ultrasonic blade defines a hooked distal portion.

According to aspects of the disclosure, a notch is defined in the distal tip of the ultrasonic blade. The notch is proximally positioned with respect to the hooked distal portion.

According to aspects of the disclosure, the distal tip of the ultrasonic blade includes a rounded distal end.

According to aspects of the disclosure, the ultrasonic blade defines a longitudinal axis. The distal tip of the ultrasonic blade defines a pointed edge facing away from the longitudinal axis of the ultrasonic blade.

According to aspects of the disclosure, the distal tip of the ultrasonic blade defines a serrated edge facing away from the longitudinal axis of the ultrasonic blade.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 illustrates a surgical system provided in accordance with the present disclosure including a surgical instrument, a surgical generator and, in some aspects, a return electrode device;

FIG. 2 is a schematic illustration of a robotic surgical system provided in accordance with the present disclosure;

FIG. 3 is a longitudinal cross-sectional view of an end effector assembly of the surgical instrument of FIG. 1;

FIG. 4 is a transverse cross-sectional view of the end effector assembly of FIG. 3;

FIG. 5A is a side view of an ultrasonic blade of an end effector assembly in accordance with the present disclosure;

FIG. 5B is a side view of the ultrasonic blade of FIG. 5A including an insulative material in accordance with the present disclosure;

FIG. 6A is a side view of an ultrasonic blade of an end effector assembly in accordance with the present disclosure;

FIG. 6B is a side view of the ultrasonic blade of FIG. 6A including an insulative material in accordance with the present disclosure;

FIG. 7A is a side view of an ultrasonic blade of an end effector assembly in accordance with the present disclosure;

FIG. 7B is a side view of the ultrasonic blade of FIG. 7A including an insulative material in accordance with the present disclosure;

FIG. 8A is a side view of an ultrasonic blade of an end effector assembly in accordance with the present disclosure;

FIG. 8B is a side view of the ultrasonic blade of FIG. 8A including an insulative material in accordance with the present disclosure;

FIG. 9A is a side view of an ultrasonic blade of an end effector assembly in accordance with the present disclosure;

FIG. 9B is a side view of the ultrasonic blade of FIG. 9A including an insulative material in accordance with the present disclosure;

FIG. 9C is a transverse cross-sectional view of the ultrasonic blade of FIG. 9A;

FIG. 10A is a sideview of an ultrasonic blade of an end effector assembly in accordance with the present disclosure; and

FIG. 10B is a sideview of the ultrasonic blade of FIG. 10A including an insulative material in accordance with the present disclosure;

### DETAILED DESCRIPTION

As used herein, the term “distal” refers to the portion that is being described which is further from a user, while the term “proximal” refers to the portion that is being described which is closer to a user. Further, to the extent consistent, any of the aspects and features detailed herein may be used in conjunction with any or all of the other aspects and features detailed herein.

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

Exemplary configurations of the disclosure will be described more fully below (e.g., with reference to the accompanying drawings). Like reference numerals may refer to like elements throughout the specification and drawings.

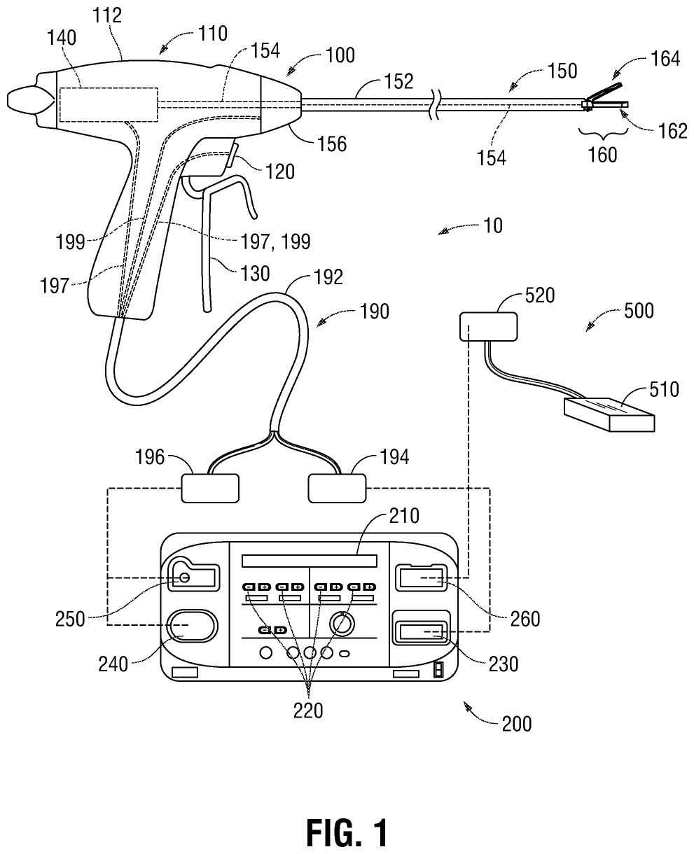

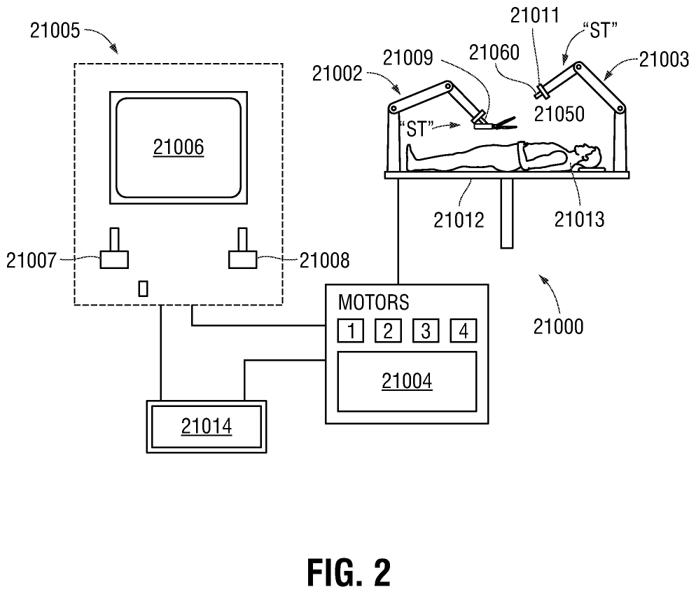

Referring to FIG. 1, a surgical system provided in accordance with aspects of the present disclosure is shown generally identified by reference numeral 10 including a surgical instrument 100, a surgical generator 200, and, in some aspects, a return electrode device 500, e.g., including a return pad 510. Surgical instrument 100 includes a handle assembly 110, an elongated assembly 150 extending distally from handle assembly 110, an end effector assembly 160 disposed at a distal end of elongated assembly 150, and a cable assembly 190 operably coupled with handle assembly 110 and extending therefrom for connection to surgical generator 200. As an alternative to handle assembly 110, surgical instrument 100 may include a robotic attachment housing for releasable engagement with a robotic arm of a robotic surgical system such as, for example, robotic surgical system 1000 (FIG. 2) detailed below.

Surgical generator 200 includes a display 210, a plurality user interface features 220, e.g., buttons, touch screens, switches, etc., an ultrasonic plug port 230, a bipolar electrosurgical plug port 240, and active and return monopolar electrosurgical plug ports 250, 260, respectively. Surgical generator 200 is configured to produce ultrasonic drive signals for output through ultrasonic plug port 230 to surgical instrument 100 to activate surgical instrument 100 in an ultrasonic mode and to provide electrosurgical energy, e.g., RF bipolar energy, for output through bipolar electrosurgical plug port 240 and/or RF monopolar energy for output through active monopolar electrosurgical port 250 to surgical instrument 100 to activate surgical instrument 100 in one or more electrosurgical modes. It is also contemplated that one or more common ports (not shown) may be configured to act as any two or more of ports 230-260. In monopolar configurations, plug 520 of return electrode device 500 is configured to connect to return monopolar electrosurgical plug port 260.

Continuing with reference to FIG. 1, handle assembly 110 includes a housing 112 defining a body portion and a fixed handle portion. Handle assembly 110 further includes an activation button 120 and a clamp trigger 130. The body portion of housing 112 is configured to support an ultrasonic transducer 140. Ultrasonic transducer 140 may be permanently engaged with the body portion of housing 112 or removable therefrom. Ultrasonic transducer 140 includes a piezoelectric stack or other suitable ultrasonic transducer components electrically coupled to surgical generator 200, e.g., via one or more of first electrical lead wires 197, to enable communication of ultrasonic drive signals to ultrasonic transducer 140 to drive ultrasonic transducer 140 to produce ultrasonic vibration energy that is transmitted along a waveguide 154 of elongated assembly 150 to blade 162 of end effector assembly 160 of elongated assembly 150, as detailed below. An activation button 120 is disposed on housing 112 and coupled to or between ultrasonic transducer 140 and/or surgical generator 200, e.g., via one or more of first electrical lead wires 197, to enable activation of ultrasonic transducer 140 in response to depression of activation button 120. In some configurations, activation button 120 may include an ON/OFF switch. In other configurations, activation button 120 may include multiple actuation switches to enable activation from an OFF position to different actuated positions corresponding to different activation settings, e.g., a first actuated position corresponding to a first activation setting and a second actuated position corresponding to a second activation setting. In still other configurations, separate activation buttons may be provided, e.g., a first actuation button for activating a first activation setting and a second activation button for activating a second activation setting.

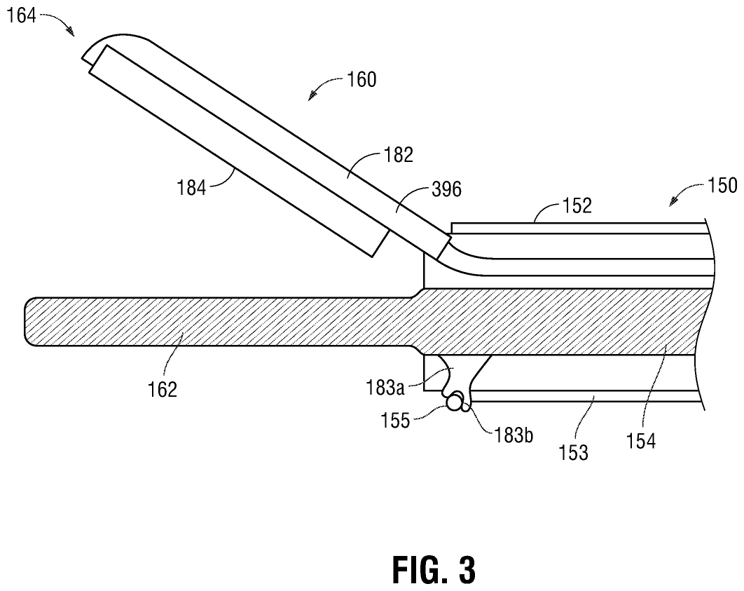

Elongated assembly 150 of surgical instrument 100 includes an outer drive sleeve 152, an inner support sleeve 153 (FIG. 3) disposed within outer drive sleeve 152, a waveguide 154 extending through inner support sleeve 153 (FIG. 3), a drive assembly (not shown), a rotation knob 156, and an end effector assembly 160 including a blade 162 and a jaw member 164. Rotation knob 156 is rotatable in either direction to rotate elongated assembly 150 in either direction relative to handle assembly 110. The drive assembly operably couples a proximal portion of outer drive sleeve 152 to clamp trigger 130 of handle assembly 110. A distal portion of outer drive sleeve 152 is operably coupled to jaw member 164 and a distal end of inner support sleeve 153 (FIG. 3) pivotably supports jaw member 164. As such, clamp trigger 130 is selectively actuatable to thereby move outer drive sleeve 152 about inner support sleeve 153 (FIG. 3) to pivot jaw member 164 relative to blade 162 of end effector assembly 160 from a spaced apart position to an approximated position for clamping tissue between jaw member 164 and blade 162. The configuration of outer and inner sleeves 152, 153 (FIG. 3) may be reversed, e.g., wherein outer sleeve 152 is the support sleeve and inner sleeve 153 (FIG. 3) is the drive sleeve. Other suitable drive structures as opposed to a sleeve are also contemplated such as, for example, drive rods, drive cables, drive screws, etc.

Referring still to FIG. 1, the drive assembly may be tuned to provide a jaw clamping force, or jaw clamping force within a jaw clamping force range, to tissue clamped between jaw member 164 and blade 162 or may include a force limiting feature whereby the clamping force applied to tissue clamped between jaw member 164 and blade 162 is limited to a particular jaw clamping force or a jaw clamping force within a jaw clamping force range.

Waveguide 154, as noted above, extends from handle assembly 110 through the inner support sleeve. Waveguide 154 includes blade 162 disposed at a distal end thereof. Blade 162 may be integrally formed with waveguide 154, separately formed and subsequently attached (permanently or removably) to waveguide 154, or otherwise operably coupled with waveguide 154. Waveguide 154 and/or blade 162 may be formed from titanium, a titanium alloy, or other suitable electrically conductive material(s), although non-conductive materials are also contemplated. Waveguide 154 includes a proximal connector (not shown), e.g., a threaded male connector, configured for engagement, e.g., threaded engagement within a threaded female receiver, of ultrasonic transducer 140 such that ultrasonic motion produced by ultrasonic transducer 140 is transmitted along waveguide 154 to blade 162 for treating tissue clamped between blade 162 and jaw member 164 or positioned adjacent to blade 162.

Cable assembly 190 of surgical instrument 100 includes a cable 192, an ultrasonic plug 194, and an electrosurgical plug 196. Ultrasonic plug 194 is configured for connection with ultrasonic plug port 230 of surgical generator 200 while electrosurgical plug 196 is configured for connection with bipolar electrosurgical plug port 240 of surgical generator 200 and/or active monopolar electrosurgical plug port 250 of surgical generator 200. In configurations where generator 200 includes a common port, cable assembly 190 may include a common plug (not shown) configured to act as both the ultrasonic plug 194 and the electrosurgical plug 196. Plural first electrical lead wires 197 electrically coupled to ultrasonic plug 194 extend through cable 192 and into handle assembly 110 for electrical connection to ultrasonic transducer 140 and/or activation button 120 to enable the selective supply of ultrasonic drive signals from surgical generator 200 to ultrasonic transducer 140 upon activation of activation button 120 in an ultrasonic mode. In addition, plural second electrical lead wires 199 are electrically coupled to electrosurgical plug 196 and extend through cable 192 into handle assembly 110. In bipolar configurations, separate second electrical lead wires 199 are electrically coupled to waveguide 154 and jaw member 164 (and/or different portions of jaw member 164) such that, as detailed below, bipolar electrosurgical energy may be conducted between blade 162 and jaw member 164 (and/or between different portions of jaw member 164). In monopolar configurations, an electrical lead wire 199 is electrically coupled to waveguide 154 such that, as also detailed below, monopolar electrosurgical energy may be supplied to tissue from blade 162. Alternatively, an electrical lead wire 199 may electrically couple to jaw member 164 in the monopolar configuration to enable monopolar electrosurgical energy to be supplied to tissue from jaw member 164. One or more second electrical lead wires 199 is electrically coupled to activation button 120 to enable the selective supply of electrosurgical energy from surgical generator 200 to waveguide 154 and/or jaw member 164 upon activation of activation button 120 in an electrosurgical mode.

As an alternative to a remote generator 200, surgical system 10 may be at least partially cordless in that it incorporates an ultrasonic generator, an electrosurgical generator, and/or a power source, e.g., a battery, thereon or therein. In this manner, the connections from surgical instrument 100 to external devices, e.g., generator(s) and/or power source(s), is reduced or eliminated.

With reference to FIG. 2, a robotic surgical system in accordance with the aspects and features of the present disclosure is shown generally identified by reference numeral 21000. For the purposes herein, robotic surgical system 21000 is generally described. Aspects and features of robotic surgical system 21000 not germane to the understanding of the present disclosure are omitted to avoid obscuring the aspects and features of the present disclosure in unnecessary detail.

Robotic surgical system 21000 generally includes a plurality of robot arms 21002, 21003; a control device 21004; and an operating console 21005 coupled with control device 21004. Operating console 21005 may include a display device 21006, which may be set up in particular to display three dimensional images; and manual input devices 21007, 21008, by means of which a person (not shown), for example a surgeon, may be able to telemanipulate robot arms 21002, 21003 in a first operating mode. Robotic surgical system 21000 may be configured for use on a patient 21013 lying on a patient table 21012 to be treated in a minimally invasive manner. Robotic surgical system 21000 may further include a database 21014, in particular coupled to control device 21004, in which are stored, for example, pre-operative data from patient 21013 and/or anatomical atlases.

Each of the robot arms 21002, 21003 may include a plurality of members, which are connected through joints, and an attaching device 21009, 21011, to which may be attached, for example, a surgical tool “ST” supporting an end effector 21050, 21060. One of the surgical tools “ST” may be ultrasonic surgical instrument 100 (FIG. 1), e.g., configured for use in both an ultrasonic mode and an electrosurgical (bipolar and/or monopolar) mode, wherein manual actuation features, e.g., actuation button 120 (FIG. 1), clamp lever 130 (FIG. 1), etc., are replaced with robotic inputs. In such configurations, robotic surgical system 1000 may include or be configured to connect to an ultrasonic generator, an electrosurgical generator, and/or a power source. The other surgical tool “ST” may include any other suitable surgical instrument, e.g., an endoscopic camera, other surgical tool, etc. Robot arms 21002, 21003 may be driven by electric drives, e.g., motors, that are connected to control device 21004. Control device 21004 (e.g., a computer) may be configured to activate the motors, in particular by means of a computer program, in such a way that robot arms 21002, 21003, their attaching devices 21009, 21011, and, thus, the surgical tools “ST” execute a desired movement and/or function according to a corresponding input from manual input devices 21007, 21008, respectively. Control device 21004 may also be configured in such a way that it regulates the movement of robot arms 21002, 21003 and/or of the motors.

Referring to FIG. 3, end effector assembly 160 of surgical instrument 100 of surgical system 10 (FIG. 1) is detailed, although end effector assembly 160 may be utilized with any other suitable surgical instrument and/or surgical system. End effector assembly 160 includes a blade 162 and a jaw member 164. Blade 162 may define a linear configuration, may define a curved configuration, or may define any other suitable configuration, e.g., straight and/or curved surfaces, portions, and/or sections; one or more convex and/or concave surfaces, portions, and/or sections; etc. With respect to curved configurations, blade 162, more specifically, may be curved in any direction relative to jaw member 164, for example, such that the distal tip of blade 162 is curved towards jaw member 164, away from jaw member 164, or laterally (in either direction) relative to jaw member 164. Further, blade 162 may be formed to include multiple curves in similar directions, multiple curves in different directions within a single plane, and/or multiple curves in different directions in different planes. In addition, blade 162 may additionally or alternatively be formed to include any suitable features, e.g., a tapered configuration, various different cross-sectional configurations along its length, cut outs, indents, edges, protrusions, straight surfaces, curved surfaces, angled surfaces, wide edges, narrow edges, and/or other features.

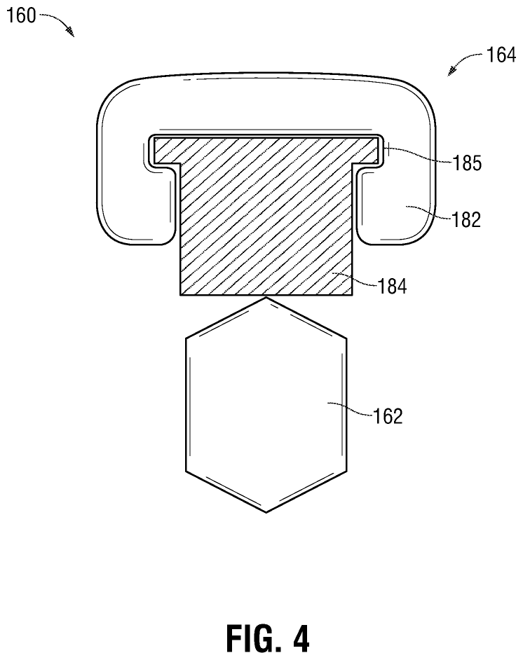

Blade 162 may define a polygonal, rounded polygonal, or any other suitable cross-sectional configuration(s) (see FIG. 4). Waveguide 154 or at least the portion of waveguide 154 proximally adjacent blade 162, may define a cylindrical shaped configuration. Plural tapered surfaces (not shown) may interconnect the cylindrically shaped waveguide 154 with the polygonal (rounded edge polygonal, or other suitable shape) configuration of blade 162 to define smooth transitions between the body of waveguide 154 and blade 162.

Blade 162 may be wholly or selectively coated with a suitable material, e.g., a non-stick material, an electrically insulative material, an electrically conductive material, combinations thereof, etc. Suitable coatings and/or methods of applying coatings include but are not limited to Teflon®, polyphenylene oxide (PPO), deposited liquid ceramic insulative coatings; thermally sprayed coatings, e.g., thermally sprayed ceramic; Plasma Electrolytic Oxidation (PEO) coatings; anodization coatings; sputtered coatings, e.g., silica; Electro Bond® coating available from Surface Solutions Group of Chicago, IL, USA: or other suitable coatings and/or methods of applying coatings.

With additional reference to FIG. 4, blade 162, as noted above, in addition to receiving ultrasonic energy transmitted along waveguide 154 from ultrasonic transducer 140 (FIG. 1), is adapted to connect to generator 200 (FIG. 1) to enable the supply of RF energy to blade 162 for conduction to tissue in contact therewith. In bipolar configurations, RF energy is conducted between blade 162 and jaw member 164 (or between portions of jaw member 164 and/or blade 162) and through tissue disposed therebetween to treat tissue. In monopolar configurations, RF energy is conducted from blade 162, serving as the active electrode, to tissue in contact therewith and is ultimately returned to generator 200 (FIG. 1) via return device 500 (FIG. 1), serving as the passive or return electrode. Alternatively, jaw member 164 (or portions thereof) may be energizable in the monopolar configuration while blade 162 is unenergized.

Jaw member 164 of end effector assembly 160 includes a more rigid structural body 182 and a more compliant jaw liner 184. Structural body 182 may be formed from an electrically conductive material, e.g., stainless steel, and/or may include electrically conductive portions. Structural body 182 includes a pair of proximal flanges 183a that are pivotably coupled to the inner support sleeve 153 via receipt of pivot bosses (not shown) of proximal flanges 183a within corresponding openings (not shown) defined within the inner support sleeve 153 and operably coupled with outer drive sleeve 152 via a drive pin 155 secured relative to outer drive sleeve 152 and pivotably received within apertures 183b defined within proximal flanges 183a. As such, sliding of outer drive sleeve 152 about inner support sleeve 153 pivots jaw member 164 relative to blade 162 from a spaced apart position to an approximated position to clamp tissue between jaw liner 184 of jaw member 164 and blade 162.

Structural body 182, or a portion(s) thereof, may be adapted to connect to a source of electrosurgical energy, e.g., generator 200 (FIG. 1), and, in a bipolar electrosurgical mode, is charged to a different potential as compared to blade 162 to enable the conduction of bipolar electrosurgical (e.g., RF) energy through tissue clamped therebetween, to treat the tissue. In a monopolar electrosurgical mode, structural body 182 may be un-energized, may be charged to the same potential as compared to blade 162 (thus both defining the active electrode), or may be energized while blade 162 is not energized (wherein structural body 182 defines the active electrode). In either monopolar configuration, energy is returned to generator 200 (FIG. 1) via return device 500 (FIG. 1), which serves as the passive or return electrode.

Jaw liner 184 is shaped complementary to a cavity 185 (FIG. 4) defined within structural body 182, e.g., defining a T-shaped configuration, to facilitate receipt and retention therein, although other configurations are also contemplated. Jaw liner 184 is fabricated from an electrically insulative, compliant material such as, for example, polytetrafluoroethylene (PTFE). The compliance of j aw liner 184 enables blade 162 to vibrate while in contact with jaw liner 184 without damaging components of ultrasonic surgical instrument 100 (FIG. 1) and without compromising the hold on tissue clamped between jaw member 164 and blade 162. Jaw liner 184 extends from structural body 182 towards blade 162 to inhibit contact between structural body 182 and blade 162 in the approximated position of jaw member 164. The insulation of jaw liner 184 maintains electrical isolation between blade 162 and structural body 182 of jaw member 164, thereby inhibiting shorting.

As detailed above, structural body 182 may be adapted to connect to a source of electrosurgical energy, e.g., generator 200 (FIG. 1), and, in a bipolar electrosurgical mode, is charged to a different potential as compared to blade 162 to enable the conduction of bipolar electrosurgical (e.g., RF) energy through tissue clamped therebetween, to treat, e.g., seal, the tissue. Additionally or alternatively, the distal portion of end effector assembly 160 (acting as a probe with jaw member 164 in the approximated position without tissue therebetween) may be advanced distally into and/or moved transversely across tissue such that tissue (and/or a conductive medium such as saline) contacts and electrically connects the distal face of blade 162 and a distal cap portion of structural body 182 to enable the conduction of bipolar electrosurgical (e.g., RF) energy through the tissue to treat or interrogate, e.g., spot coagulate, cut, or otherwise treat, the tissue.

Referring particularly to FIGS. 3 and 4, end effector assembly 160 is described in accordance with configurations of the present disclosure. The end effector assembly 160 includes the ultrasonic blade 162 adapted to receive ultrasonic energy to vibrate the ultrasonic blade 162. The jaw member 164 is movable relative to the ultrasonic blade 162 from a spaced-apart position to an approximated position for clamping tissue. The jaw member 164 includes the structural body 182. A jaw liner 184 is engaged with the structural body 182 such that the jaw liner 184 contacts the ultrasonic blade 162 when the jaw member 164 is in the approximated position.

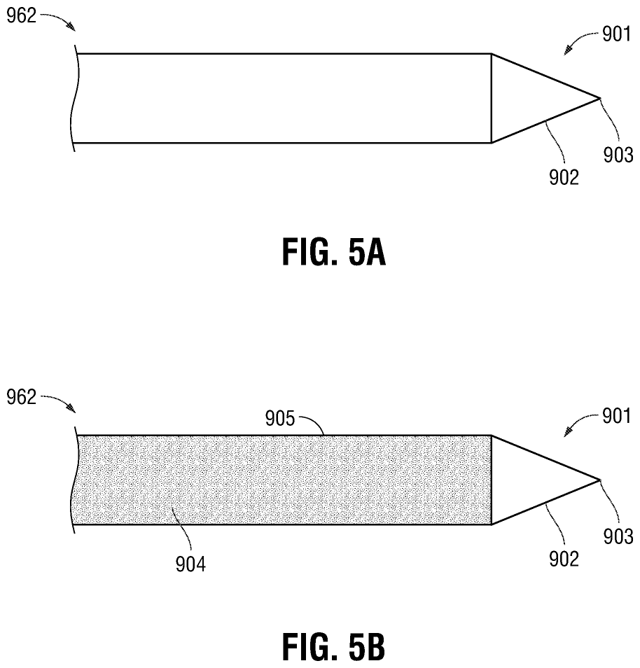

Various ultrasonic blades employable in an end effector assembly of a surgical instrument are described below with reference to FIGS. 5A-10B. Unless otherwise indicated below, the end effector assembly employing the ultrasonic blades described below is substantially the same as the end effector assemblies described above with reference to FIGS. 1, 3, and 4. Unless otherwise indicated below, the insulative coatings described above (e.g., with respect to ultrasonic blade 162) may be substantially the same as the insulative coatings described below.

Referring to FIG. 5A, an ultrasonic blade 962 is described. The ultrasonic blade 962 is adapted to receive ultrasonic energy from a source of ultrasonic energy, e.g., ultrasonic energy transmitted along waveguide 154 from ultrasonic transducer 140 (FIG. 1), to vibrate the ultrasonic blade 962. The ultrasonic blade 962 is adapted to receive electrosurgical energy from a source of electrosurgical energy, e.g., generator 200 (FIG. 1). The ultrasonic blade 962 defines a distal tip 901. The distal tip 901 of the ultrasonic blade 962 includes an edge 902 defining a taper and a pointed distal end 903. For example, distal tip 901 may define a conical shape.

According to aspects of the disclosure, the ultrasonic blade 962 is adapted for monopolar treatment. As an example, the ultrasonic blade 962 is adapted to receive ultrasonic energy and electrosurgical (RF) energy substantially simultaneously, consecutively, independently, or in any other suitable manner. Ultrasonic blade 962 may also be adapted for bipolar treatment.

Referring to FIG. 5B, a portion of the ultrasonic blade 962 includes a coating 904 adapted to direct monopolar energy. The coating 904 may be an insulative coating and/or a nonstick coating. As an example, the coating 904 extends along a portion 905 of the ultrasonic blade 962 proximal to the distal tip 901. The coating 904 directs monopolar energy toward the tapered edge 902 and/or the pointed distal end 903 of the ultrasonic blade 962, thus focusing the tissue treatment to the area adjacent distal tip 901.

Any features described for ultrasonic blade 962 are similarly available to ultrasonic blades 1062, 1162, 1262, 1362 and 1462 described below.

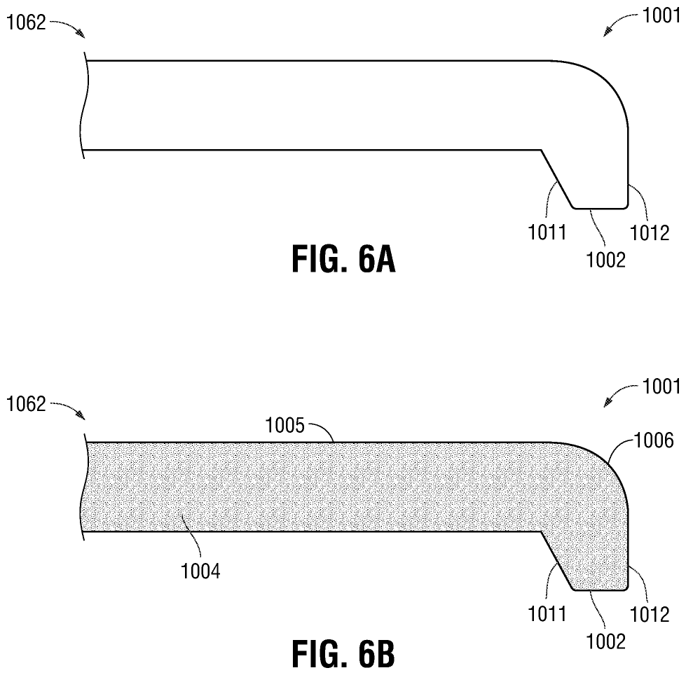

Referring to FIG. 6A, an ultrasonic blade 1062 is described. The ultrasonic blade 1062 defines a distal tip 1001. The distal tip 1001 of the ultrasonic blade 1062 defines a hooked distal portion 1002. The hooked distal portion 1002 may be configured to apply monopolar treatment on a proximal-facing surface 1011 or a distal-facing surface 1012 of the ultrasonic blade 1062.

Referring to FIG. 6B, a portion of the ultrasonic blade 1062 includes a coating 1004, e.g., an insulative and/or non-stick coating, adapted to direct monopolar energy. As an example, the coating 1004 extends along a portion 1005 of the ultrasonic blade 1062 proximal to the distal tip 1001 and extends partially into a proximal region 1006 of the distal tip 1001. The coating 1004 directs monopolar energy toward the hooked distal portion 1002 of the ultrasonic blade 1062. As an example, monopolar energy is directed to the proximal-facing surface 1011 and/or the distal-facing surface 1012 of the ultrasonic blade 1062.

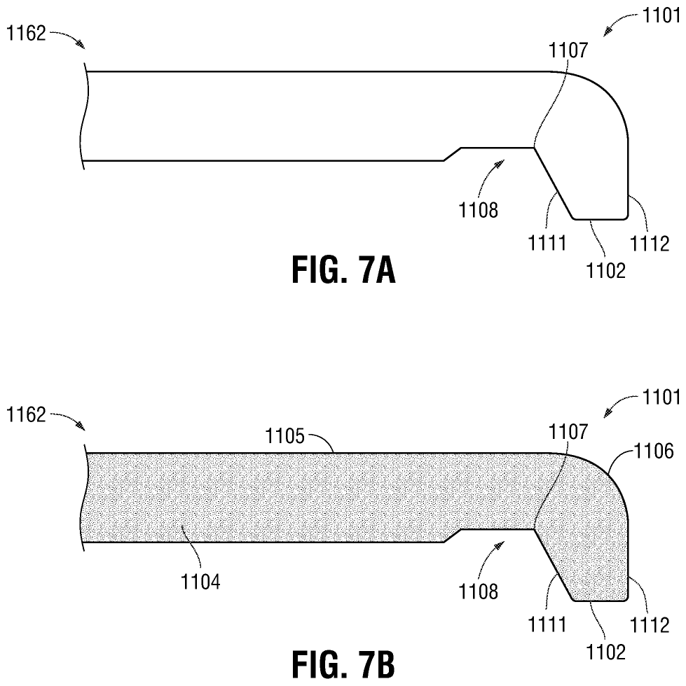

Referring to FIG. 7A, an ultrasonic blade 1162 is described. The ultrasonic blade 1162 defines a distal tip 1101. The distal tip 1101 of the ultrasonic blade 1162 defines a hooked distal portion 1102 defining a notch 1108 at a proximal side 1107 of the hooked distal portion 1102. The proximal side 1107 of distal tip 1101 may define a sharp edge (e.g., an edge suitable for mechanically cutting tissue) facing an inner area of notch 1108. The hooked distal portion 1102 may be configured to apply monopolar treatment on a proximal-facing surface 1111 and/or a distal-facing surface 1112 of the ultrasonic blade 1162. Monopolar treatment may also be applied by the ultrasonic blade 1162 in the notch 1108 of the ultrasonic blade 1162. Hooked distal portion 1102 may reside in a same diameter or envelope as the ultrasonic blade 1162. Alternatively, hooked distal portion 1102 may extend outside and protrude from an envelope of the ultrasonic blade 1162. For example, in use, the notch 1108 and the proximal-facing surface 1111 of the ultrasonic blade 1162 may be employed for monopolar treatment of tissue as the ultrasonic blade 1162 is retracted in a proximal direction.

Referring to FIG. 7B, a portion of the ultrasonic blade 1162 includes a coating 1104 adapted to direct monopolar energy. As an example, the coating 1104 extends along a portion 1105 of the ultrasonic blade 1162 proximal to the distal tip 1101 and extends partially into a proximal region 1106 of the distal tip 1101. The coating 1104 directs monopolar energy toward the notch 1108 of the ultrasonic blade 1162 and/or to the proximal-facing surface 1111 of the ultrasonic blade 1162.

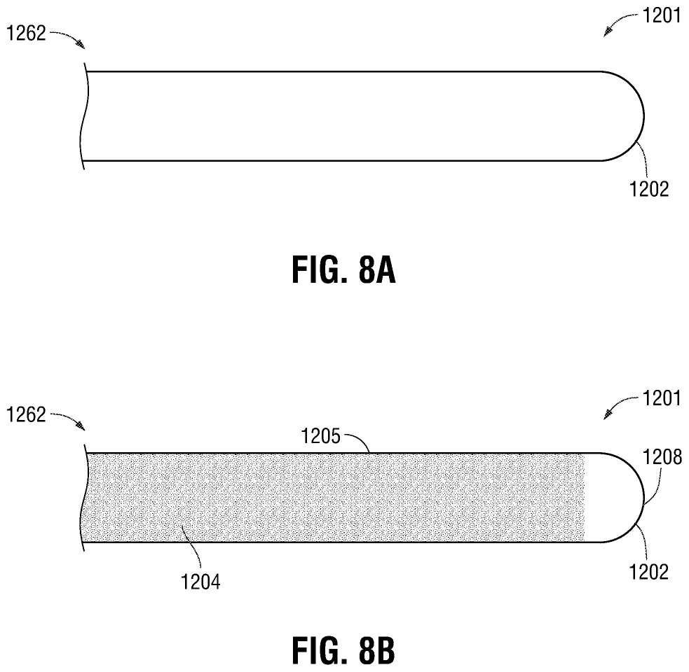

Referring to FIG. 8A, an ultrasonic blade 1262 is described. The ultrasonic blade 1262 defines a distal tip 1201. The distal tip 1201 of the ultrasonic blade 1262 defines a curved distal portion 1202 including a rounded distal-facing edge 1208.

Referring to FIG. 8B, a portion of the ultrasonic blade 1262 includes a coating 1204 adapted to direct monopolar energy. As an example, the coating 1204 extends along a portion 1205 of the ultrasonic blade 1262 proximal to the distal tip 1201. The coating 1204 directs monopolar energy toward the curved distal portion 1202.

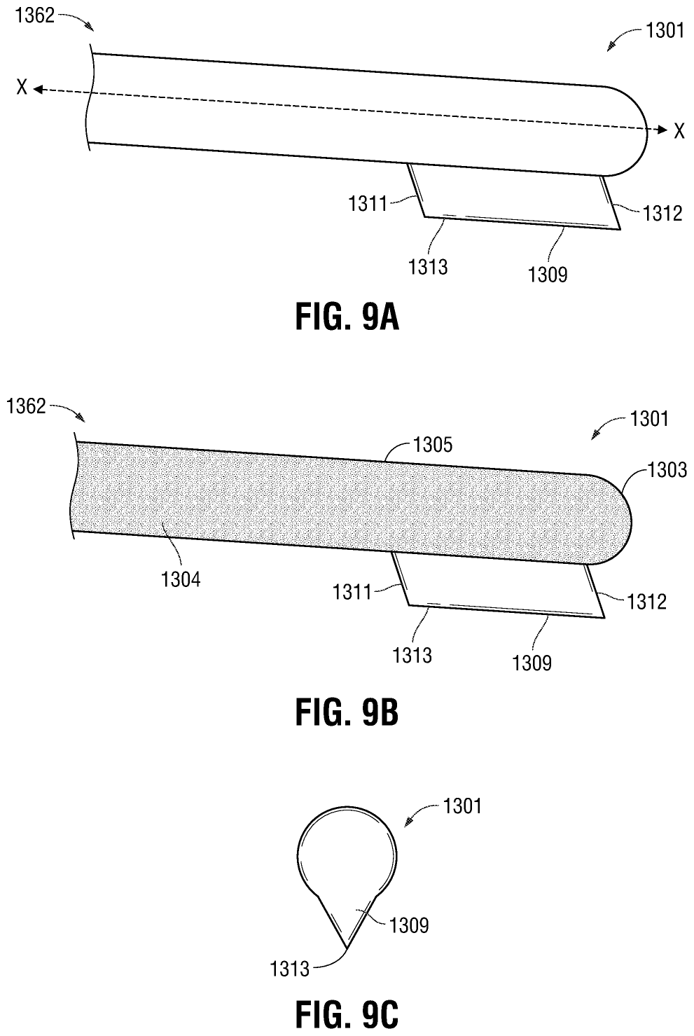

Referring to FIGS. 9A and 9C, an ultrasonic blade 1362 defining a longitudinal axis x-x is described. The ultrasonic blade 1362 defines a distal tip 1301. The distal tip 1301 of the ultrasonic blade 1362 defines a pointed edge 1309 facing away from the longitudinal axis x-x of the ultrasonic blade 1362. The distal tip 1301 may define a teardrop shape in which a portion of the pointed edge 1309 closer to the longitudinal axis x-x is wider than surface 1313 opposite the longitudinal axis x-x (see, e.g., FIG. 9C). The pointed edge 1309 may be configured to apply monopolar treatment on a proximal-facing surface 1311, a distal-facing surface 1312 and/or the surface 1313 facing away from the longitudinal axis x-x of the ultrasonic blade 1362.

Referring to FIG. 9B, a portion of the ultrasonic blade 1362 includes a coating 1304 adapted to direct monopolar energy. As an example, the coating 1304 extends from a proximal portion 1305 of the ultrasonic blade 1362 to a distal end 1303 of the ultrasonic blade 1362. The coating 1304 directs monopolar energy toward the pointed edge 1309 of the ultrasonic blade 1362. As an example, monopolar energy is directed to the proximal-facing surface 1311, the distal-facing surface 1312 and/or the surface 1313 facing away from the longitudinal axis x-x of the ultrasonic blade 1362. In use, the pointed edge 1309 may be employed for applying monopolar energy by advancing the ultrasonic blade 1362 in a distal direction, a proximal direction, or in a direction substantially perpendicular to the longitudinal axis x-x.

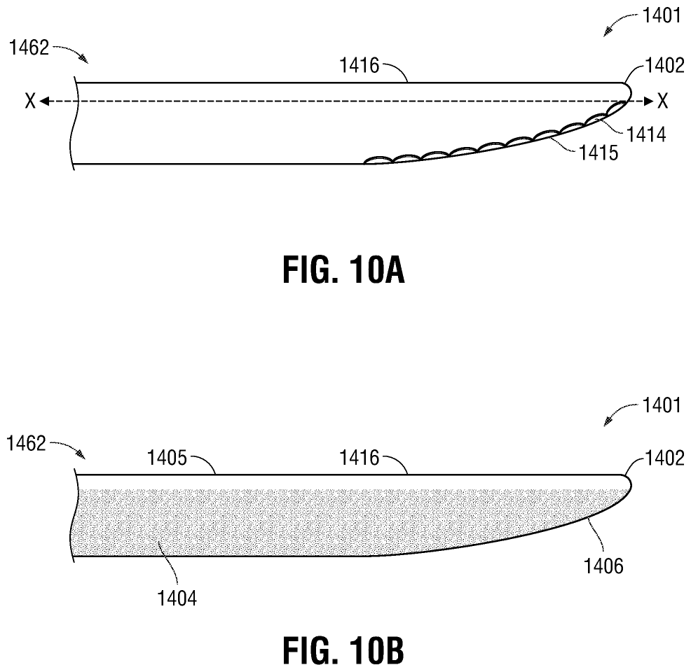

Referring to FIG. 10A, an ultrasonic blade 1462 defining a longitudinal axis x-x is described. The ultrasonic blade 1462 defines a distal tip 1401. The distal tip 1401 of the ultrasonic blade 1462 defines a serrated edge 1414 facing away from the longitudinal axis x-x of the ultrasonic blade 1462. The serrated edge 1414 is configured for backscoring. Monopolar treatment may be applied by the distal tip 1401 on any of a bottom surface 1415, a top surface 1416 or a distal end 1402 of the ultrasonic blade 1462.

Referring to FIG. 10B, a portion of the ultrasonic blade 1462 includes a coating 1404 adapted to direct monopolar energy. As an example, the coating 1404 extends along a portion 1405 of the ultrasonic blade 1462 proximal to the distal tip 1401 and extends partially into a proximal region 1406 of the distal tip 1401. The coating 1404 directs monopolar energy toward the top surface 1416 and the distal end 1402 of the ultrasonic blade 1062. Alternatively or additionally, the serrated edge 1414 may remain uncoated to facilitate direction of monopolar energy thereto.

The above-detailed configurations of ultrasonic blades including coatings (see, e.g., FIGS. 5B, 6B, 7B, 8B, 9B, and 10B) may be reversed; that is, where the indicated portions are un-coated and the remainder of the ultrasonic blade is coated. The coatings in such configurations may be non-stick, insulative, or conduction-enhanced coatings, e.g., to facilitate monopolar energy conduction therefrom. Likewise, the indicated and unindicated portions may both be coated with coatings having different properties, e.g., insulative versus conduction-enhancing, etc.

While several aspects of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. An end effector assembly of a surgical instrument, comprising:
an ultrasonic blade adapted to receive ultrasonic energy from a source of ultrasonic energy to vibrate the ultrasonic blade; and
a jaw member movable relative to the ultrasonic blade from a spaced-apart position to an approximated position for clamping tissue therebetween; and
a jaw liner engaged with the jaw member such that the jaw liner contacts the ultrasonic blade when the jaw member is in the approximated position,
wherein the ultrasonic blade is adapted to receive electrosurgical energy from a source of electrosurgical energy, and
wherein the ultrasonic blade defines a distal tip, the distal tip of the ultrasonic blade including one of:
a tapered edge defining a pointed distal end;
a hooked portion;
a rounded distal end;
a pointed edge facing away from the longitudinal axis of the ultrasonic blade; or
a serrated edge facing away from the longitudinal axis of the ultrasonic blade.

2. The end effector assembly according to claim 1, wherein the ultrasonic blade is adapted for monopolar treatment or bipolar treatment.

3. The end effector assembly of claim 1, wherein the ultrasonic blade is adapted to receive ultrasonic energy and electrosurgical energy simultaneously.

4. The end effector assembly of claim 1, wherein a portion of the ultrasonic blade includes an insulative coating adapted to direct monopolar energy.

5. The end effector assembly of claim 4, wherein the insulative coating is a nonstick coating.

6. The end effector assembly of claim 4, wherein the insulative coating is not disposed on the distal tip of the ultrasonic blade.

7. The end effector assembly of claim 6, wherein the insulative coating covers the entirety of the ultrasonic blade except for the distal tip of the ultrasonic blade.

8. The end effector assembly of claim 1, wherein the distal tip includes the hooked portion and wherein the ultrasonic blade further includes a notch defined in the distal tip, the notch proximally positioned with respect to the hooked portion.
